(19) United States
(12) Patent Application Publication
(10) Pub. No.: US 2012/0142988 A1
(43) Pub. Date: Jun. 7, 2012

(54) METHOD OF PRODUCING 1-HEXENE

(75) Inventor: Shinjiro Ishihara, Ichihara (JP)

(73) Assignee: SUMITOMO CHEMICAL COMPANY, LIMITED, Chuo-ku, Tokyo (JP)

(21) Appl. No.: 13/388,911

(22) PCT Filed: Aug. 5, 2009
(86) PCT No.: PCT/JP2010/063591

§ 371 Date: Feb. 3, 2012

(30) Foreign Application Priority Data
Aug. 5, 2009 (JP) 2009-182206

(51) Int. Cl. C07C 2/30; C07C 7/00

(52) U.S. Cl. 585/512

(57) ABSTRACT

A method of producing 1-hexene comprising:
step 1: a periodic table group IV transition metal compound, an organoaluminum compound and ethylene are brought into contact with each other in a solvent to obtain a solution containing 1-hexene; and
step 2: the solution obtained in step 1 is brought into contact with an aqueous solution containing a metal hydroxide and having a pH of 12.0 or higher.

### FIELD OF THE INVENTION

The present invention relates to a method of producing 1-hexene, more particularly to a producing method of 1-hexene which can efficiently remove metallic components at a state of a good oil-water separatability and is capable of preventing corrosion of equipment.

### BACKGROUND OF THE INVENTION

1-hexene is used as a base material for linear low-density polyethylenes and the like. Regarding the production methods of 1-hexene, for instance Patent Document 1 discloses a method in which ethylene is trimerized by using a specific titanium complex and an organoaluminum oxy compound as catalysts.
[Patent Document 1] WO 09/5003

### BRIEF SUMMARY OF THE INVENTION

In the meantime, in the industrial and continuous production of 1-hexene, since metallic components are contained in a reaction liquid, problems of e.g. an adhesion of the metallic components to a distillation column can be brought about with depending upon the conditions of distillation and separation of 1-hexene. Additionally, when a residual solution, after a separation of 1-hexene from a reaction liquid, is recycled and reused or is disposed, if metallic components are contained in residual solution, in case of recycling and reusing, there is a possibility that the metallic components are precipitated as fouling components in an inside, inlet port or exhaust port etc. of a pipe or pump to come not to be able to conduct a long period of operation. In case that the residual solution is disposed, there is a possibility that the metallic components are precipitated as fouling components on a burner nozzle etc. at the burnout of waste oil etc. to come not to be able to conduct a long period of operation.

As mentioned above, in the industrial and continuous production of 1-hexene, it is important to remove metallic components contained in a reaction liquid.

For example, Patent Document 1 discloses that the reaction solution containing the produced 1-hexene is washed with hydrochloric acid solution and pure water.

The method described in Patent Document 1, however, had the problem of causing corrosion of the equipment because of use of acids.

The theme of the present invention is to provide a method of producing 1-hexene which can efficiently remove metallic components at a state of a good oil-water separatability and is capable of preventing corrosion of equipment.

The present inventors accomplished the present invention by finding that metallic components can be efficiently removed at a state of a good oil-water separatability by using alkalis having specific conditions.

More specifically, the present invention relates to a method of producing 1-hexene comprising,

step 1: a periodic table group IV transition metal compound, an organoaluminum compound and ethylene are brought into contact with each other in a solvent to obtain a solution containing 1-hexene; and

step 2: the solution obtained in step 1 is brought into contact with an aqueous solution containing a metal hydroxide and having a pH of 12.0 or higher.

According to the present invention, it is possible to efficiently remove metallic components at a state of a good oil-water separatability and to prevent corrosion of equipment in the process of producing 1-hexene.

### DETAILED DESCRIPTION OF THE INVENTION

The method of producing 1-hexene according to the present invention comprises a step in which a periodic table group IV transition metal compound, an organoaluminum compound and ethylene are brought into contact with each other in a solvent to obtain a solution containing 1-hexene (this step may hereinafter be referred to as “step 1”) and a step in which the solution obtained in step 1 is brought into contact with an aqueous solution containing a metal hydroxide and having a pH of 12.0 or higher (this step may hereinafter be referred to as “step 2”).

### [Step 1]

Step is 1 is a step in which a periodic table group IV transition metal compound, an organoalminum compound and ethylene are brought into contact with each other in a solvent to form a solution containing 1-hexene. Step 1 preferably comprises contacting a periodic table group IV transition metal compound, an organoaluminum compound and ethylene in a solvent to form a solution containing 1-hexene produced at a selectivity of 50% or higher. Here, selectivity is calculated by:

(weight (g) of 1-hexene obtained in step 1)/(weight (g) of ethylene consumed in step 1).

As the periodic table group IV transition metal compound used in step 1, it is possible to use, for instance, titanium compounds, zirconium compounds and hafnium compounds. Of these compounds, titanium compounds are preferred in view of their catalytic activity and hexene selectivity.

As such titanium compounds, those represented by the following general formula [1] are cited as examples:

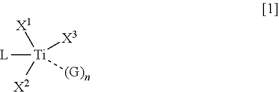

(wherein L represents a C5-C30 group having a cyclopentadiene type anionic skeleton or a C6-C30 aryloxy group; X¹, X² and X³ may be identical or different and represent independently a hydrogen atom, a halogen atom, a C1-C30 hydrocarbyl group, a C1-C30 hydrocarbyloxy group, a C2-C30 hydrocarbylcarbonyloxy group or a carbamoyl group; G represents a neutral ligand; n is an integer of 0 to 2, and when n is 2, the two neutral ligands may be identical or different).

The C5-C30 groups having a cyclopentadiene type anionic skeleton and the C6-C30 aryloxy groups represented by L and the C1-C30 hydroxdycarbyl groups, C1-C30 hydrocarbyloxy groups, C2-C30 hydroxycarbylcarbonyloxy group and carbamoyl groups represented by X¹, X² and X³ may have a substituent.

The halogen atom represented by X¹, X² and X³ may be, for instance, fluorine atom, chlorine atom, bromine atom and iodine atom.

Examples of the C1-C30 hydrocarbyl groups represented by X¹, X² and X³ are C1-C30 alkyl groups, C2-C30 alkenyl groups, C2-C30 alkynyl groups, C3-C30 cycloalkyl groups, C5-C30 cycloalkenyl groups, C6-C30 aryl groups and C7-C30 aralkyl groups.

Examples of the C1-C30 alkyl groups include methyl group, ethyl group, n-propyl group, isopropyl group, n-butyl group, sec-butyl group, tert-butyl group, n-pentyl group, neopentyl group, isoamyl group, n-hexyl group, n-octyl group, n-decyl group, n-dodecyl group, n-pentadecyl group and n-eicosyl group.

As examples of the C2-C30 alkenyl groups are cited vinyl group, allyl group, propenyl group, 2-methyl-2-propenyl group, homoallyl group, pentenyl group, hexenyl group, heptenyl group, octenyl group, nonenyl group and decenyl group.

Examples of the C2-C30 alkynyl groups are ethynyl group and propargyl group.

Examples of the C3-C30 cycloalkyl groups include cyclopropyl group, cyclobutyl group, cyclopentyl group, cyclohexyl group and adamantyl group.

Exemplary of the C5-C30 cycloalkenyl groups are cyclopentadienyl group, indenyl group and fluorenyl group.

Examples of the C6-C30 aryl groups include phenyl group, 2-tolyl group, 3-tolyl group, 4-tolyl group, 2,3-xylyl group, 2,4-xylyl group, 2,5-xylyl group, 2,6-xylyl group, 3,4-xylyl group, 3,5-xylyl group, 2,3,4-trimethylphenyl group, 2,3,5-trimethylphenyl group, 2,3,6-trimethylphenyl group, 2,4,6-trimethylphenyl group, 3,4,5-trimethylphenyl group, 2,3,4,5-tetramethylphenyl group, 2,3,4,6-tetramethylphenyl group, 2,3,5,6-tetramethylphenyl group, pentamethylphenyl group, ethylphenyl group, n-propylphenyl group, isopropylphenyl group, n-butylphenyl group, sec-butylphenyl group, tert-butylphenyl group, n-pentylphenyl group, neopentylphenyl group, n-hexylphenyl group, n-octylphenyl group, n-decylphenyl group, n-dodecylphenyl group, n-tetradecylphenyl group, naphthyl group, and anthracenyl group.

Examples of the C7-C30 aralkyl groups include benzyl group, (2-methylphenyl)methyl group, (3-methylphenyl)methyl group, (4-methylphenyl)methyl group, (2,3-dimethylphenyl)methyl group, (2,4-dimethylphenyl)methyl group, (2,5-dimethylphenyl)methyl group, (2,6-dimethylphenyl)methyl group, (3,4-dimethylphenyl)methyl group, (3,5-dimethylphenyl)methyl group, (2,3,4-trimethylphenyl)methyl group, (2,3,5-trimethylphenyl)methyl group, (2,3,6-trimethylphenyl)methyl group, (3,4,5-trimethylphenyl)methyl group, (2,4,6-trimethylphenyl)methyl group, (2,3,4,5-tetramethylphenyl)methyl group, (2,3,4,6-tetramethylphenyl)methyl group, (2,3,5,6-tetramethylphenyl)methyl group, (pentamethylphenyl)methyl group, (ethylphenyl)methyl group, (n-propylphenyl)methyl group, (isopropylphenyl)methyl group, (n-butylphenyl)methyl group, (sec-butylphenyl)methyl group, (tert-butylphenyl)methyl group, (n-pentylphenyl)methyl group, (neopentylphenyl)methyl group, (n-hexylphenyl)methyl group, (n-octylphenyl)methyl group, (n-decylphenyl)methyl group, naphthylmethyl group, and anthracenylmethyl group.

The C1-C30 hydrocarbyl groups may have a substituent such as, for example, a halogen atom and a C1-C30 hydroxycarbyloxy group. Examples of such a halogen atom are fluorine atom, chlorine atom, bromine atom and iodine atom, and examples of the C1-C30 hydrocarbyloxy groups are C1-C30 alkoxyl and C6-C30 aryloxy groups.

Examples of the C1-C30 hydrocarbyloxy groups represented by X¹, X² or X³ include C1-C30 alkoxyl groups, C6-C30 aryloxy groups, and C7-C30 aralkyloxy groups.

Examples of the C1-C30 alkoxy groups include methoxy group, ethoxy group, n-propoxy group, isopropoxy group, n-butoxy group, sec-butoxy group, t-butoxy group, n-pentoxy group, neopentoxy group, n-hexoxy group, n-octoxy group, n-dodesoxy group, n-pentadesoxy group, and n-icosoxy group.

Examples of the C6-C30 aryloxy groups include phenoxy group, 2-methylphenoxy group, 3-methylphenoxy group, 4-methylphenoxy group, 2,3-dimethylphenoxy group, 2,4-dimethylphenoxy group, 2,5-dimethylphenoxy group, 2,6-dimethylphenoxy group, 3,4-dimethylphenoxy group, 3,5-dimethylphenoxy group, 2,3,4-trimethylphenoxy group, 2,3,5-trimethylphenoxy group, 2,3,6-trimethylphenoxy group, 2,4,5-trimethylphenoxy group, 2,4,6-trimethylphenoxy group, 3,4,5-trimethylphenoxy group, 2,3,4,5-tetramethylphenoxy group, 2,3,4,6-tetramethylphenoxy group, 2,3,5,6-tetramethylphenoxy group, pentamethylphenoxy group, ethylphenoxy group, n-propylphenoxy group, isopropylphenoxy group, n-butylphenoxy group, sec-butylphenoxy group, tert-butylphenoxy group, n-hexylphenoxy group, n-octylphenoxy group, n-decylphenoxy group, n-tetradecylphenoxy group, naphthoxy group, and anthracenoxy group and, 6-adamantyl-4-methyl-2-[N-{2-(2-methoxyphenyl)}phenyl]iminophenoxy group.

Examples of the C7-C30 aralkyloxy groups include benzyloxy group, (2-methylphenyl)methoxy group, (3-methylphenyl)methoxy group, (4-methylphenyl)methoxy group, (2,3-dimethylphenyl)methoxy group, (2,4-dimethylphenyl)methoxy group, (2,5-dimethylphenyl)methoxy group, (2,6-dimethylphenyl)methoxy group, (3,4-dimethylphenyl)methoxy group, (3,5-dimethylphenyl)methoxy group, (2,3,4-trimethylphenyl)methoxy group, (2,3,5-trimethylphenyl)methoxy group, (2,3,6-trimethylphenyl)methoxy group, (2,4,5-trimethylphenyl)methoxy group, (2,4,6-trimethylphenyl)methoxy group, (3,4,5-trimethylphenyl)methoxy group, (2,3,4,5-tetramethylphenyl)methoxy group, (2,3,4,6-tetramethylphenyl)methoxy group, (2,3,5,6-tetramethylphenyl)methoxy group, (pentamethylphenyl)methoxy group, (ethylphenyl)methoxy group, (n-propylphenyl)methoxy group, (isopropylphenyl)methoxy group, (n-butylphenyl)methoxy group, (sec-butylphyenyl)methoxy group, (tert-butylphenyl)methoxy group, (n-hexylphenyl)methoxy group, (n-octylphenyl)methoxy group, (n-decylphenyl)methoxy group, naphthylmethoxy group, and anthracenylmethoxy group.

The C1-C30 hydrocarbyloxy groups may have a substituent, for instance, a halogen atom such as fluorine atom, chlorine atom, bromine atom or iodine atom.

Exemplary of the C2-C30 hydrocarbylcarbonyloxy groups represented by X¹, X² or X³ are C2-C30 alkylcarbonyloxy groups, C7-C30 arylcarbonyloxy groups, and C8-C30 aralkylcarbonyloxy groups.

Examples of the C2-C30 alkylcarbonyloxy groups include methylcarbonyloxy group, ethylcarbonyloxy group, n-propylcarbnyloxy group, isopropylcarbonyloxy group, n-butylcarbonyloxy group, sec-butylcarbonyloxy group, tert-butylcarbonyloxy group, n-pentylcarbonyloxy group, neopentylcarbonyloxy group, isoamylcarbonyloxy group, n-hexylcarbonyloxy group, n-octylcarbonyloxy group, n-decylcarbonyloxy group, n-dodecylcarbonyloxy group, n-pentadecylcarbonyloxy group, and n-eicosylcarbonyloxy group.

Examples of the C7-C30 arylcarbonyloxy groups include phenylcarbonyloxy group, 2-tolylcarbonyloxy group, 3-tolylcarbonyloxy group, 4-tolylcarbonyloxy group, 2,3-xylylcarbonyloxy group, 2,4-xylylcarbonyloxy group, 2,5-xylylcarbonyloxy group, 2,6-xylylcarbonyloxy group, 3,4-xylylcarbonyloxy group, 3,5-xylylcarbonyloxy group, 2,3,4-trimethylphenylcarbonyloxy group, 2,3,5-trimethylphenylcarbonyloxy group, 2,3,6-trimethylphenylcarbonyloxy group, 2,4,6-trimethylphenylcarbonyloxy group, 3,4,6-trimethylphenylcarbonyloxy group, 2,3,4,5-tetramethylphenylcarbonyloxy group, 2,3,4,6-tetramethylphenylcarbonyloxy group, 2,3,5,6-tetramethylphenylcarbonyloxy group, and pentamethylphenylcarbonyloxy group.

Examples of the C8-C30 aralkylcarbonyloxy groups include benzylcarbonyloxy group, (2-methylphenyl)methylcarbonyloxy group, (3-methylphenyl)methylcarbonyloxy group, (4-methylphenyl)methylcarbonyloxy group, (2,3-dimethylphenyl)methylcarbonyloxy group, (2,4-dimethylphenyl)methylcarbonyloxy group, (2,5-dimethylphenyl)methylcarbonyloxy group, (2,6-dimethylphenyl)methylcarbonyloxy group, (3,4-dimethylphenyl)methylcarbonyloxy group, (3,5-dimethylphenyl)methylcarbonyloxy group, (2,3,4-trimethylphenyl)methylcarbonyloxy group, (2,3,5-trimethylphenyl)methylcarbonyloxy group, (2,3,6-trimethylphenyl)methylcarbonyloxy group, (3,4,5-trimethylphenyl)methylcarbonyloxy group, (2,4,6-trimethylphenyl)methylcarbonyloxy group, (2,3,4,5-tetramethylphenyl)methylcarbonyloxy group, (2,3,4,6-tetramethylphenyl)methylcarbonyloxy group, and (2,3,5,6-tetramethylphenyl)methylcarbonyloxy group.

The C2-C30 hydrocarbylcarbonyloxy groups may have a substituent. The substituent may, for instance, be a halogen atom such as fluorine atom, chlorine atom, bromine atom or iodine atom.

The carbamoyl groups represented by X¹, X² or X³ may have a substituent, for example, a C1-C30 hydrocarbyl group.

The neutral ligand represented by G may be, for instance, a compound having nitrogen atoms, a compound having oxygen atoms, a compound having phosphorus atoms and a compound having sulfur atoms. Examples of the compounds having nitrogen atoms are amine compounds, amide compounds and nitrile compounds. Examples of the compounds having oxygen atoms are ether compounds, ketone compounds, aldehyde compounds and ester compounds. Examples of the compounds having phosphorus atoms are trialkylphosphine compounds, triarylphosphine compounds and phosphine oxide compounds. Examples of the compounds having sulfur atoms are thioether compounds, and sulfone compounds. More specifically, the above-mentioned ligand may be, for instance, pyridine, tetramethylethylenediamine, tetrahydrofuran, diethyl ether, triphenylphosphine, dimethyl sulfide or thiophene.

Cyclopentadienyl groups, indenyl groups and fluorenyl groups may be cited as examples of the C5-C30 groups having a cyclopentadiene type anionic skeleton represented by L. The C5-C30 groups having a cyclopentadiene type anionic skeleton represented by L may have a substituent, for example, a C1-C30 alkyl group and a C3-C30 trialkylsilyl group.

As examples of the C6-C30 aryloxy groups also represented by L, the same examples as shown above in relation to X¹, X² and X³ can be cited.

The C5-C30 groups having a cyclopentadiene type anionic skeleton represented by L are preferably the groups represented by the following general formula [2]:

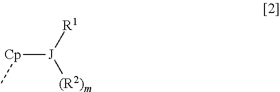

(wherein Cp---- indicates linkage to Ti; Cp represents a C5-C30 group having a cyclopentadiene type anionic skeleton; J represents a periodic table group XIII-XVI atom; R¹ represents a C6-C30 aryl group or a C7-C30 aralkyl group; R² represents a hydrogen atom or a C1-C30 hydrocarbyl group; m indicates the valence of J-2; when m is an integer or 2 or greater, R²s may be same or different and may be combined to form a ring).

Exemplary of the C5-C30 groups having a cycopentdiene type anionic skeleton represented by Cp are cyclopentadienyl group, indenyl group and fluorenyl group. The C5-C30 groups having a cycopentdiene type anionic skeleton represented by Cp may have a substituent, for example, a C1-C30 alkyl group and a C3-C30 trialkylsilyl group.

The periodic table group IIIX-XVI atom represented by J may be, for instance, boron atom, carbon atom, silicon atom, nitrogen atom, phosphorus atom, oxygen atom or sulfur atom. Carbon atom or silicon atom is preferred.

As examples of the C6-C30 aryl groups and C7-C30 aralkyl groups represented by R¹ and the C1-C30 hydrocarbyl groups represented by R², the same examples as shown above with reference to X¹, X² and X³ can be cited.

The C6-C30 aryloxy groups represented by L are preferably those having the structure represented by the following general formula [3] or [4]:

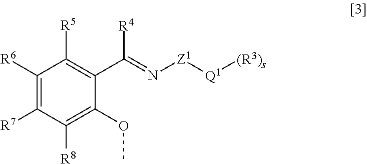

(wherein O---- indicates linkage to Ti; R³ to R⁸ may be identical or different and represent independently a hydrogen atom, a halogen atom, a C1-C30 hydrocarbyl group, a C1-C30 hydrocarbyloxy group, a heterocyclic compound residue, a C2-C30 dihydrocarbylamino group or a C1-C30 hydrocarbylsilyl group; two or more of R³ to R⁸ may combine to form a ring; s is a number of 1 or 2, and when s is 2, two R^{a}s may be same or different; Q¹ represents an oxygen atom, nitrogen atom, phosphorus atom or sulfur atom; Z¹ represents a C1-C30 hydrocarbon group or a heterocyclic compound residue; the form of bond linking Z¹ and Q¹ may be single bond, double bond or triple bond; the bond linking Q¹ and R³ may be also single bond, double bond or triple bond; R³ and Z¹ may combine to form a ring.)

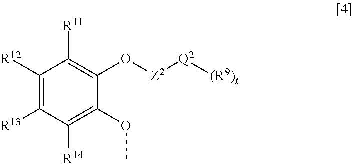

(wherein O---- indicates linkage to Ti; R⁹ to R¹⁴ may be identical or different, each representing independently a hydrogen atom, a halogen atom, a C1-C30 hydrocarbyl group, a C1-C30 hydrocarbyloxy group, a heterocyclic compound residue, a C2-C30 dihydrocarbylamino group or a C1-C30 hydrocarbylsilyl group, two or more of these groups may combine to form a ring; t indicates a number of 1 or 2, and when t is 2, two R⁹s may be same or different; Q² represents an oxygen atom, nitrogen atom, phosphorus atom or sulfur atom; Z² represents a C1-C30 hydrocarbon group or a heterocyclic compound residue; the bond linking Z² and Q² may be single bond, double bond or triple bond; also the bond linking Q² and R⁹ may be single, double or triple bond; R⁹ and Z² may combine to form a ring.)

As examples of the halogen atoms, C1-C30 hydrocarbyl groups and C1-C30 hydrocarbyloxy groups represented by R³ to R¹⁴, the same examples as shown with reference to X¹, X² and X³ can be cited.

As the heterocyclic compounds of the heterocyclic compound residues represented by R³ to R¹⁴, pyrrole, pyridine, pyrimidine, quinoline, triazine, furan, pyran and thiophene can be cited as examples. These heterocyclic compounds may have a substituent the examples of which are halogen atoms, C1-C30 hydrocarbyl groups and C1-C30 hydrocarbyloxy groups.

The C2-C30 dihydrocarbylamino groups represented by R³ to R¹⁴ are those amino groups who have two hydrogen atoms thereon substituted with two hydrocarbyl groups. Exemplary of the above-mentioned hydrocarbyl groups are C1-C15 alkyl groups, C3-C15 cycloalkyl groups and C6-C15 aryl groups. Examples of the C1-C15 alkyl groups include methyl group, ethyl group, n-propyl group, isopropyl group, n-butyl group, sec-butyl group, tert-butyl group, isobutyl group, n-pentyl group and h-hexyl group. Cyclohexyl group can be cited as an example of the C3-C15 cycloalkyl groups, and phenyl group can be cited as an example of the C6-C15 aryl groups. Examples of such C2-C30 dihydrocarbylamino groups include dimethylamino group, diethylamino group, di-n-propylamino group, diisopropylamino group, di-n-butylamino group, di-sec-butylamino group, di-tert-butylamino group, di-isobutylamino group, tert-butylisopropylamino group, di-n-hexylamino group, di-n-octylamino group, di-n-decylamino group, diphenylamino group, bis(trimethylsilyl)amino group, and bis(tert-butyldimethylsilyl)amino group.

The C1-C30 hydrocarbylsilyl groups represented by R³ to R¹⁴ are those silyl groups which have one to three hydrogen atoms thereon substituted with one to three hydrocarbyl groups.

Examples of such hydrocarbyl groups are C1-C30 alkyl groups, C3-C30 cycloalkyl groups and C6-C30 aryl groups. Examples of the C1-C30 alkyl group include methyl group, ethyl group, n-propyl group, isopropyl group, n-butyl group, sec-butyl group, tert-butyl group, isobutyl group, n-pentyl group and n-hexyl group. Cyclohexyl group can be cited as an example of the C3-C30 cycloalkyl groups, and phenyl group can be cited as an example of the C6-C30 aryl groups. Exemplary of such C1-C30 hydrocarbylsilyl groups are C1-C30 monohydrocarbylsilyl groups, C2-C30 dihydrocarbylsilyl groups and C3-C30 trihydrocarbylsilyl groups. Examples of the C1-C30 monohydrocarbylsilyl groups are methylsilyl groups, ethylsilyl groups and phenylsilyl groups, and examples of the C2-C30 dihydrocarbylsilyl groups are dimethylsilyl groups, diethylsilyl groups and diphenylsilyl groups. Examples of the C3-C30 trihydrocarbylsilyl groups include trimethylsilyl groups, triethylsilyl groups, tri-n-propylsilyl groups, triisopropylsiyl groups, tri-n-butylsilyl groups, tri-sec-butylsilyl groups, tri-tert-butylsilyl groups, tri-isobutylsilyl groups, tert-butyl-dimethylsilyl groups, tri-n-pentylsilyl groups, tri-n-hexylsilyl groups, tricyclohexylsilyl groups, and triphenylsilyl groups.

Any of these hydrocarbylsilyl groups may have its hydrocarbyl group substituted with a halogen atom, for instance, a fluorine, chlorine, bromine or iodine atom.

The C1-C30 hydrocarbon groups represented by Z¹ and Z² are exemplified by C1-C30 alkylene groups, C2-C30 alkenylene groups and C6-C30 arylene groups.

Examples of the C1-C30 alkylene groups include methylene group, methylmethylene group, ethylene group, ethylmethylene group, propylene group, propylmethylene group, butylene group, butylmethylene group, pentylene group, pentylmethylene group, dimethylmethylene group, methylethylmethylene group, methylpropylmethylene group and methylbutylmethylene group. Examples of the C2-C30 alkenylene groups include vinylene group, propenylene group, butenylene group, pentenylene group and butadienylene group. Examples of the C6-C30 arylene groups include phenylene group, naphthylene group, biphenylene group, anthranylene group and xylylene group. The C1-C30 hydrocarbon groups may have a substituent which may, for instance, be a halogen atom or a C1-C30 hydrocarbyloxy group.

As the heterocyclic compounds of the heterocyclic compound residues represented by Z¹ and Z², pyrrole, pyridine, pyrimidine, quinoline, triazine, furan, pyran and thiophene can be cited as examples. These heterocyclic compounds may have one or more substituent the examples of which are halogen atoms, C1-C30 hydrocarbyl groups and C1-C30 hydrocarbyloxy groups.

In view of feasibility of producing objective 1-hexene with high selectivity, atomicity of Z¹ which links Q¹ and N is preferably 3 to 5 in the compounds represented by the general formula [3], while atomicity of Z² linking Q² and O is preferably 4 or 5 in the compounds represented by the general formula [4].

Atomicity of Z¹ linking Q¹ and N or atomicity of Z² linking Q² and O can be counted in the manner illustrated in (A) or (B) below. In the case of (A), atomicity is 3, and in the case of (B), it is 4.

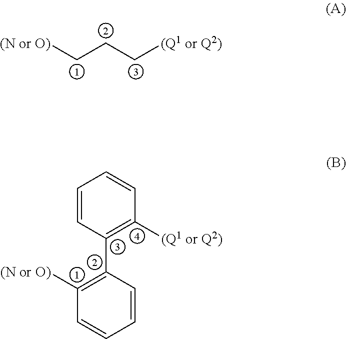

The periodic table group IV transition metal compounds include, as examples thereof, cyclopentadienyltitanium trichloride, pentamethylcyclopentadienyltitanium trichloride, indenyltitanium trichloride, fluorenyltitanium trichloride, (1-methyl-1-phenyl)ethylcyclopentadienyltitanium trichloride, 6-adamantyl-4-methyl-2-[N-{2-(2-methoxyphenyl)}phenyl]iminophenoxytitanium tricloride, and the compounds obtained by converting these chlorides into methyl, methoxide, phenoxide, benzyloxide, acetate, dimethylamide, hydride, bromide or iodide. Preferred in these compounds are (1-methyl-1-phenyl)ethylcyclopentadienyltitanium trichloride, and 6-adamantyl-4-methyl-2-[N-{2-(2-methoxyphenyl)}phenyl]iminophenoxytitaniuim trichloride. More preferred is 6-adamantyl-4-methyl-2-[N-{2-(2-methoxyphenyl)}imino-phenoxytitanium trichloride.

As the organoaluminum compound used in step 1, trialkylaluminum compounds, alkylaluminum halides, alkylaluminum hydrides, aluminoxane compounds and the like can be cited as examples. Examples of the trialkylaluminum compounds are trimethylaluminum, triethylaluminum, tri-n-butylaluminum, triisobutylaluminum and tri-n-octylaluminum. Exemplary of the alkylaluminum halides are diethylaluminum chloride and ethylaluminum dichloride. Diethylaluminum hydride can be cited as a typical example of the alkylaluminum hydrides, and methylaluminoxane can be named as an example of the aluminoxane compounds. Of these compounds, methylaluminoxane is preferred in view of its activity and selectivity. These organoaluminum compounds can be used either singly or as a mixture of two or more of them.

The organoaluminum compounds may be used by supporting them on a carrier. As carrier, there can be used, for instance, metal oxides, complex oxides of metals and inorganic halides. The metal oxides include among their examples silica, magnesia, alumina and titania. The complex oxides of metals include silica-alumina, and silica-titania. Magnesium chloride and magnesium bromide can be cited as examples of the inorganic halides.

As the solvent used in step 1, linear saturated hydrocarbon compounds, alicyclic saturated hydrocarbon compounds, aromatic hydrocarbon compounds, and linear unsaturated hydrocarbon compounds can be cited as examples. The linear saturated hydrocarbon compounds are preferably those with a carbon number of 4 to 10, more preferably butane, pentane, hexane, heptane or octane, even more preferably butane, pentane, hexane or heptane, particularly preferably butane, pentane or heptane. The alicyclic saturated hydrocarbon compounds are preferably those with a carbon number of 4 to 10, more preferably cyclohexane, methylcyclohexane or decalin, even more preferably cyclohexane or methylcyclohexane, particularly preferably cyclohexane. The aromatic hydrocarbon compounds are preferably those with a carbon number of 6 to 10, more preferably benzene, toluene, xylene, ethylbenzene, mesitylene or tetralin, even more preferably benzene, toluene or xylene, particularly preferably toluene. The linear unsaturated hydrocarbon compounds are preferably those with a carbon number of 4 to 10, more preferably 1-hexene. These solvents can be used either singly or as a mixture of two or more of them.

The solvent used in step 1 is preferably selected from butane, pentane hexane, heptane, cyclohexane, methylcyclohexane, benzene, toluene, xylene and 1-hexene, more preferably selected from butane, pentane, heptane, cyclohexane, toluene and 1-hexene.

The reaction operation in step 1 is carried out at a temperature of preferably 0 to 200° C., under a pressure preferably between normal pressure and 20 MPa for a period of preferably one minute to 20 hours. The reaction operation in step 1 may be either batch-wise, semi-batch-wise or continuous.

The weight ratio of the periodic table group IV transition metal compound to the organoaluminum compound used in step 1 (weight (g) of periodic table group IV transition metal compound/weight (g) of organoaluminum compound) is preferably between 0.0001 and 10.

In the present invention, in step 1, in order to inhibit the side reactions from occurring in the course of step 1, preferably a periodic table group IV transition metal compound, an organoaluminum compound and ethylene are brought into contact with each other in a solvent to form a reaction mixture, and this reaction mixture is further contacted with an alcohol to obtain a solution containing 1-hexene.

The term “alcohol” is used here to refer to the compounds having at least one alcoholic hydroxyl group, the examples thereof being methanol, ethanol, 1-propanol, 2-propanol, 1-butanol, 2-butanol, sec-butanol, t-butanol, 1-hexanol, 1-octanol, 2-ethyl-1-hexanol and benzyl alcohol.

### [Step 2]

Step 2 is a step in which the solution obtained in step 1 is brought into contact with an aqueous solution containing a metal hydroxide and having a pH of 12.0 or higher (this aqueous solution may hereinafter be referred to as “alkaline aqueous solution”).

Contact between the solution obtained in step 1 and an aqueous solution containing a metal hydroxide and having a pH of 12.0 or higher (alkaline aqueous solution) in step 2 may be conducted at an appropriate stage. For instance, the solution obtained in step 1 may be brought into contact with an aqueous solution containing a metal hydroxide and having a pH of 12.0 or higher (alkaline aqueous solution), or the solution obtained after separating at least part of 1-hexene from the solution obtained in step 1 may be brought into contact with an aqueous solution containing a metal hydroxide and having a pH of 12.0 or higher (alkaline aqueous solution).

The alkaline aqueous solution used in step 2 is an aqueous solution containing a metal hydroxide and having a pH of 12.0 or higher. By the term “metal hydroxide” are meant here the metal compounds containing at least one hydroxyl group. Examples of such compounds are lithium hydroxide, sodium hydroxide, potassium hydroxide, rubidium hydroxide, cesium hydroxide, calcium hydroxide. Sodium hydroxide and potassium hydroxide are preferred in view of easy industrial availability.

In the present invention, in the solution obtained in step 1 are contained, in addition to 1-hexene, the metallic components of the periodic table group IV transition metal compound and the organoaluminum compound used in step 1. In order to remove these metallic components into the aqueous layer, it is essential to use an aqueous solution containing a metal hydroxide and having a pH of 12.0 or higher in step 2.

When no metal hydroxide is contained or the pH of the aqueous solution is less than 12.0, separatability between the solution containing 1-hexene and the aqueous layer containing the metallic components is unsatisfactory, hindering the operation of separating the solution containing 1-hexene from the aqueous layer containing the metallic components to hinder the removal of the metallic components.

A variety of extracting means can be used in bringing the solution obtained in step 1 into contact with the alkaline aqueous solution. Preferably a combination of a stirring vessel and a static separation vessel is used and the solution obtained in step 1 is brought into contact with the alkaline aqueous solution, and then a separation of a water layer (aqueous layer) and an oil layer is conducted. In use of such a combination of stirring vessel and static separation vessel, their combination may be either single- or multiple-staged. Also, the extraction operation may be either batch-wise or continuous.

In performing contact of the solution obtained in step 1 with an alkaline aqueous solution and then conducting a separation of a water layer and an oil layer by using a stirring vessel and a static separation vessel, the weight ratio of the solution obtained in step 1 to the alkaline aqueous solution is given by: weight (g) of the solution obtained in step 1/weight (g) of the alkaline aqueous solution =0.01 to 100. This operation is conducted at a temperature of usually 25 to 60° C. for a period of usually 5 to 120 minutes.

In the present invention, the solution obtained in step 1 may contain polyethylene as a by-product in addition to 1-hexene, so that a step for removing polyethylene from the step 1 solution may be provided between step 1 and step 2. A method for removing polyethylene from the step 1 solution comprises using such means as filter and centrifugal separator. Also, in the present invention, an additional step for removing the solvent from the step 1 solution may be provided between step 1 and step 2. Distillation is suggested as a method for removing the solvent from the step 1 solution.

In the present invention, the water layer, which is obtained after bringing into contacting with the alkaline aqueous solution, is an alkaline solution containing metallic components. The alkaline solution can be reused as an alkaline aqueous solution used in step 2. When the alkaline solution is reused, since metallic components are accumulated, a part (together with the metallic components) of the alkaline solution is removed to an outside of system and simultaneously remaining alkaline solution is reused and a fresh alkaline aqueous solution is added in an amount corresponding to an amount of the removed alkaline solution to be used.

In the present invention, since the metallic components can be removed to a water layer by step 2, when 1-hexene is separated, in case of conducting a distillation, problems of an adhesion of the metallic components to a distillation column are not brought about. Additionally, when a residual solution, after a separation of 1-hexene, is recycled and reused, the metallic components are not precipitated as fouling components in an inside, inlet port or exhaust port etc. of a pipe or pump to come to be able to conduct a long period of operation. In case that the residual solution is disposed, the metallic components are not precipitated as fouling components on a burner nozzle etc. at the burnout of waste oil etc. to come to be able to conduct a long period of operation.

The process for producing 1-hexene according to the present invention is preferably:
(1) A method comprising a step in which a periodic table group IV transition metal compound, an organoaluminum compound and ethylene are brought into contact with each other in a solvent to obtain a solution containing 1-hexene (step 1), a step in which the solution obtained in step 1 is brought into contact with an aqueous solution containing a metal hydroxide and having a pH of 12.0 or higher to separate out a solution containing 1-hexene and a water layer (this step may hereinafter be referred to as “step 2′”), and a step for separating 1-hexene from the 1-hexene-containing solution obtained in step 2′ (this step may hereinafter referred to as “step 3”; or
(2) A method comprising a step in which a periodic table group IV transition metal compound, an organoaluminum compound and ethylene are brought into contact with each other in a solvent to obtain a solution containing 1-hexene (step 1), a step in which at least part of 1-hexene is separated from the solution obtained in step 1 to obtain 1-hexene or a solution containing 1-hexene and a residual solution after removal of at least part of 1-hexene (this step may hereinafter referred to as “step 4”), and a step in which the residual solution obtained in step 4 is brought into contact with an aqueous solution containing a metal hydroxide and having a pH of 12.0 or higher to effect oil-water separation (this step may hereinafter be referred to as “step 2″”).

[Step 2′]

Step 2′ is a process in which the solution obtained in step 1 is brought into contact with an aqueous solution containing a metal hydroxide and having a pH of 12.0 or higher (alkaline aqueous solution) to separate out a solution containing 1-hexene and a water layer.

As the alkaline aqueous solution used in step 2′, the same as exemplified as the alkaline aqueous solutions for use in step 2 described above can be cited as examples.

In the present invention, in the solution obtained in step 1 are contained, beside 1-hexene, metal components deriving from the periodic table group IV transition metal compound and the organoaluminum compound used in step 1, In order to remove these metallic components into the water layer, it is necessary in step 2′ to use an aqueous solution containing a metal hydroxide and having a pH of 12.0 or higher

When no metal hydroxide is contained or the pH of the aqueous solution is less than 12.0, separatability between the solution containing 1-hexene and the aqueous layer containing the metallic components proves unsatisfactory, hindering the operation of separating the solution containing 1-hexene and the aqueous layer containing the metallic components and hindering the removal of the metallic components.

A variety of extracting means can be used in contacting the solution obtained in step 1 and the alkaline aqueous solution. Preferably a combination of stirring vessel and static separation vessel are used. When using such a stirring vessel-static separation vessel combination, their combination may be either single- or multiple-staged. Also, the extraction operation may be either batch-wise or continuous.

In performing contact of the solution obtained in step 1 with an alkaline aqueous solution and causing separation into a solution containing 1-hexene and a water layer by using a stirring vessel and a static separation vessel, the weight ratio of the solution obtained in step 1 to the alkaline aqueous solution is given by: weight (g) of the solution obtained in step 1/weight (g) of the alkaline aqueous solution=0.01 to 100. This operation is conducted at a temperature of usually 25 to 60° C. for a period of usually 5 to 120 minutes.

In the present invention, the solution obtained in step 1 may contain polyethylene as a by-product in addition to 1-hexene, so that a step for removing polyethylene from the step 1 solution may be provided between step 1 and step 2′. A method for removing polyethylene from the step 1 solution comprises using such means as filter and centrifugal separator. Also, in the present invention, an additional step for removing the solvent from the step 1 solution may be provided between step 1 and step 2′. Distillation is suggested as a method for removing the solvent from the step 1 solution. The solvent removed can be reused as a solvent to be used in step 1.

### [Step3]

Step 3 is a step in which 1-hexene is separated from the solution containing 1-hexene obtained in step 2′. As for the rate of separation of 1-hexene, it is preferable to separate 50 wt % or more, more preferably 60 wt % or more, even more preferably 70 wt % or more of 1-hexene yielded in step 1.

Distillation is a method for separating 1-hexene from the solution given in step 1. Alkaline components may be admixed in the solution containing 1-hexene obtained in step 2′. Before conducting a distillation, the alkaline components may be removed with a water washing. Additionally, by a distillation, in case of recovering 1-hexene, a solvent contained in the solution may be simultaneously separated, and the solvent separated may be reused as a solvent to be used in step 1.

In case that the present invention is a method of producing 1-hexene comprising step 1, step 2′ and step 3, since the metallic components can be removed to a water layer by step 2′, when 1-hexene is separated, in case of conducting a distillation, problems of an adhesion of the metallic components to a distillation column are not brought about. Additionally, when a residual solution, after a recovery of 1-hexene at step 3, is recycled and reused, the metallic components are not precipitated as fouling components in an inside, inlet port or exhaust port etc. of a pipe or pump to come to be able to conduct a long period of operation. In case that the residual solution is disposed, the metallic components are not precipitated as fouling components on a burner nozzle etc. at the burnout of waste oil etc. to come to be able to conduct a long period of operation.

### [Step 4]

Step 4 is an operation for separating at least part of 1-hexene from the solution obtained in step 1 to obtain 1-hexene or a solution containing 1-hexene and a residual solution left after separation of at least part of 1-hexene.

A method for separating 1-hexene from the solution obtained in step 1 is distillation.

When 1-hexene is separated by distillation from the solution obtained in step 1, the solvent contained in the solution may be separated at the same time. The solvent removed can be reused as a solvent to be used in step 1.

The expression “at least part of 1-hexene is separated” used here means weather that the whole amount of 1-hexene may be separated from the step 1 solution or that part of 1-hexene may be separated, with part of 1-hexene being allowed to remain in the residual solution.

As for the rate of separation of 1-hexene, it is preferable to separate 50 wt % or more, more preferably 60 wt % or more, even more preferably 70 wt % or more of 1-hexene yielded in step 1.

### [Step 2″]

Step 2″ is a process in which the residual solution obtained in step 4 and an aqueous solution containing a metal hydroxide and having a pH of 12.0 or higher (alkaline aqueous solution) are brought into contact to perform oil-water separation.

As the alkaline aqueous solution used in step 2″, it is possible to use the same alkaline aqueous solutions as mentioned for use in step 1.

In the residual solution obtained in step 4, there are contained metallic components deriving from the periodic table group IV transition metal compound and the organoaluminum compound used in step 1. In step 4, in order to remove these metal components into the water layer, it needs to apply an aqueous solution containing a metal hydroxide and having a pH of 12.0 or above. When no metal hydroxide is contained or the pH of the aqueous solution is less than 12.0, separatability between the organic layer and the water layer is unsatisfactory, hindering the operation of separating these two layers and hindering the removal of the metallic components.

A variety of extracting means can be used in contacting the residual solution obtained in step 4 and an alkaline aqueous solution and conducting oil-water separation. Preferably a combination of stirring vessel and static separation vessel are used. When employing such a stirring vessel-static separation vessel combination, their combination may be either single- or multiple-staged. Also, the extraction operation may be either batch-wise or continuous.

In performing contact of the residual solution obtained in step 4 with an alkaline aqueous solution and conducting oil-water separation by using a stirring vessel and a static separation vessel, the weight ratio of the solution obtained in step 4 to the alkaline aqueous solution is usually given by: weight (g) of the solution obtained in step 1/weight (g) of the alkaline aqueous solution=0.01 to 100. This operation is conducted at a temperature of usually 25 to 60° C. for a period of usually 5 to 120 minutes.

The oil layer obtained in step 2″ contains a solvent which can be recovered from the oil layer by distilling to be able to be reused as a solvent to be used in step 1. Alkaline components may be admixed in the oil layer. Before conducting a distillation, the alkaline components may be removed with a water washing.

The water layer obtained by step 2″ is an alkaline solution containing metallic components. The alkaline solution can be reused as an alkaline aqueous solution used in step 2. When the alkaline solution is reused, since metallic components are accumulated, a part (together with the metallic components) of the alkaline solution is removed to an outside of system and simultaneously remaining alkaline solution is reused and a fresh alkaline aqueous solution is added in an amount corresponding to an amount of the removed alkaline solution to be used.

In case that the present invention is a method of producing 1-hexene comprising step 1, step 4 and step 2″, since the metallic components can be removed to a water layer by step 2″, when the oil layer, after conducting oil-water separation at step 2″, is recycled and reused, the metallic components are not precipitated as fouling components in an inside, inlet port or exhaust port etc. of a pipe or pump to come to be able to conduct a long period of operation. In case that the oil layer is disposed, the metallic components are not precipitated as fouling components on a burner nozzle etc. at the burnout of waste oil etc. to come to be able to conduct a long period of operation.

### EXAMPLES

The present invention is further illustrated by its embodiments.

### Referential Example 1

0.0005 mmol of a titanium complex (0.5 mM toluene solution) represented by the following formula (1), 5 mmol of methylaluminoxane (MMAO-3A made by TOSO Finechem, 1 M hexane solution) and 144 mL of toluene are mixed, and then 3.2 MPa of ethylene is introduced, reacting the mixture at 25° C. for 60 minutes. After the reaction, 50 mmol of 2-ethyl-1-hexanol is introduced to stop the reaction. Ethylene is depressurized to obtain a reaction solution. The reaction solution contains 0.0005 mmol of a titanim complex represented by the following formula (1), 5 mmol of methylaminoxane, 144 mL of toluene, 5 mL of hexane, 50 mmol of 2-ethyl-1-hexanol, 2.85 g of decenes, and 0.57 g of polyethylene.

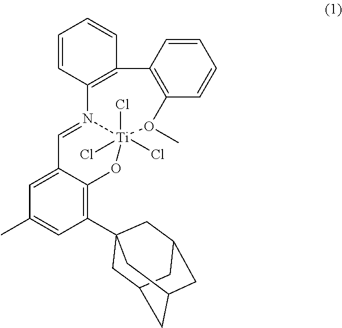

### Referential Example 2

The reaction solution obtained in Referential Example 1 is filtered to separate polyethylene, giving a filtrate comprising 0.0005 mmol of the titanium complex represented by the above-shown formula (1), 5 mmol of methylaluminoxane, 144 mL of toluene, 5 mL of hexane, 50 mmol of 2-ethyl-1-hexanol, 53.58 g of 1-hexene and 2.85 g of decenes.

### Example 1

### (1) Preparation of Reaction Model Oil

0.43 g of 1 mM toluene solution of the titanium complex of the above-shown formula (1), 2.80 g of methylaminoxane (MMAO-3A made by TOSO Finechem, hexane solution containing 5.4 wt % of Al), 123.88 g of toluene, 5.97 g of 2-ethyl-1-hexanol and 4.14 g of 1-decene were mixed. This mixed solution was used as a reaction model oil.

(2) Contact with Alkaline Aqueous Solution

7.03 g of the reaction model oil prepared in (1) above, 3.07 g of 1-hexene and 4.97 g of a sodium hydroxide solution with pH 13.3 were fed into a 20 mL screw-capped bottle having a stirrer tip therein, stirred vigorously for 5 minutes and then allowed to stand for 2 minutes. As a result, it could be confirmed that the mixed solution separated into two layers.

### Referential Example 3

From the filtrate obtained in Referential Example 2,1-hexene and hexane were removed by distillation, obtaining a distillation residue. This distillation residue containes 0.0005 mmol of the titanium complex represented by the above-shown formula (1), 5 mmol of methylaminohexane, 144 mL of toluene, 50 mmol of 2-ethyl-1-hexanol, and 2.85 g of decenes.

### Example 2

10.02 g of the reaction model oil prepared in (1) of Example 1 and 5.05 g of a sodium hydroxide solution with pH 13.3 were supplied into a 20 mL screw-capped bottle having a stirrer tip, stirred vigorously for 5 minutes and then allowed to stand for 2 minutes. As a result, it could be confirmed that the mixed solution separated into two layers

### Comparative Example 1

The same experiment as conducted in Example 2 was carried out except that 5.01 g of a sodium hydroxide solution with pH 11.8 was used instead of 5.05 g of a sodium hydroxide solution with pH 13.3. As a result, a cloudy layer was observed present between the two layers.

### Comparative Example 2

The same experiment as conducted in Example 2 was carried out except that 10.01 g of the reaction model oil and 5.01 g of an ammonia solution with pH 13.3 were used in place of 10.02 g of the reaction model oil and 5.05 g of a sodium hydroxide solution with pH 13.3. As a result, a cloudy layer was seen present between the two layers.

### Comparative Example 3

The same experiment as conducted in Example 2 was carried out except that 9.99 g of the reaction model oil and 5.09 g of a pH 11.0 sodium carbonate solution were used in place of 10.02 g of the reaction model oil and 5.05 g of a pH 13.3 sodium hydroxide solution. As a result, a cloudy layer was observed present between the two layers.

### Example 3

The same experiment as conducted in Example 2 was carried out except that 1.02 g of the reaction model oil and 5.02 g of a pH 13.3 sodium hydroxide solution were used in place of 10.02 g of the reaction model oil and 5.05 g of a pH 13.3 sodium hydroxide solution. As a result, it could be confirmed that the mixed solution separated into two layers.

### Example 4

The same experiment as conducted in Example 2 was carried out except that 0.11 g of the reaction model oil and 5.17 g of a pH 13.3 sodium hydroxide solution were used in place of 10.02 g of the reaction model oil and 5.05 g of a pH 13.3 sodium hydroxide solution. As a result, it could be confirmed that the mixed solution separated into two layers.

### Example 5

The same experiment as conducted in Example 2 was carried out except that 10.06 g of the reaction model oil and 0.50 g of a pH 13.3 sodium hydroxide solution were used in place of 10.02 g of the reaction model oil and 5.05 g of a pH 13.3 sodium hydroxide solution. As a result, it was confirmed that the mixed solution separated into two layers.

### Example 6

(1) Production of 1-hexene

In a well argon-substituted 200 mL autoclave equipped with a stirrer, 5.5 mmol of methylaminoxane (MMAO-3A made by TOSO Finechem, 1 M hexane solution) and 95 mL of toluene were mixed, and then 3.2 MPa of ethylene was introduced. After additionally introducing 0.0005 mmol of a titanium complex (1.0 mM toluene solution) represented by the following formula (1), the mixture was stirred for 30 minutes at 40° C. After the reaction, 1 mL of 2-ethyl-1-hexanol was introduced to end the reaction. After cooling the reaction mixture, ethylene was depressurized to obtain a reaction solution A. This reaction solution contained 7.7 wt % of 1-hexene and 0.17 wt % of aluminum. Titanium was unmeasurable as it was below the detection limit.

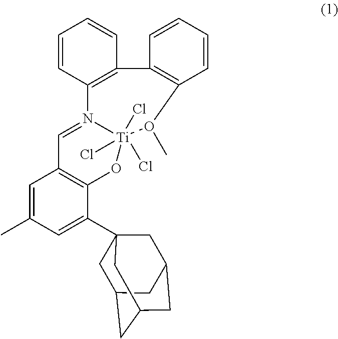

(2) Contacting with an Alkaline Aqueous Solution

20.04 g of the reaction solution A and 10.01 g of a sodium hydroxide solution with pH 12.8 were supplied into a 50 mL screw-capped bottle having a stirrer tip, vigorously stirred for 5 minutes and then allowed to stand for 2 minutes. As a result, it could be confirmed that the mixed solution separated into two layers. Substantially no aluminum and titanium were contained in the oil layer after separation.

### Comparative Example 4

The same experiment as conducted in Example 6 (2) was carried out except that 19.99 g of the reaction solution A and 10.00 g of a sodium hydroxide solution with pH 11.1 were used in place of 20.04 g of the reaction solution A and 10.01 g of a sodium hydroxide solution with pH 12.8. As a result, a cloudy layer was observed present between the two layers. Since it was difficult to collect the oil layer, the metallic concentration was not able to be analyzed.

### Comparative Example 5

The same experiment as conducted in Example 6 (2) was carried out except that 20.03 g of the reaction solution A and 10.01 g of a sodium hydroxide solution with pH 13.3 were used in place of 20.04 g of the reaction solution A and 10.01 g of a sodium hydroxide solution with pH 12.8. As a result, a cloudy layer was observed present between the two layers. Since it was difficult to collect the oil layer, the metallic concentration was not able to be analyzed.

### Example 7

(1) Production of 1-hexene

In a well argon-substituted 200 mL autoclave equipped with a stirrer, 5.5 mmol of methylaminoxane (MMAO-3A made by TOSO Finechem, 1 M hexane solution) and 95 mL of toluene were mixed, and then 3.2 MPa of ethylene was introduced. After additionally introducing 0.0005 mmol of a titanium complex (1.0 mM toluene solution) represented by the following formula (1), the mixture was stirred for 30 minutes at 40° C. After the reaction, 1 mL of 2-ethyl-1-hexanol was introduced to end the reaction. After cooling the reaction mixture, ethylene was depressurized to obtain a reaction solution A′. This reaction solution contained 5.4 wt % of 1-hexene.

(2) Separation of 1-hexene

The reaction solution A′ was distilled under normal pressure to separate 1-hexene. The solution which remained after separation of 1-hexene was called residual solution B. 1.1 wt % of 1-hexene and 0.16 wt % of aluminum was contained in the residual solution B. Titanium was unmeasurable as it was below the detection limit.

(3) Contacting with an Alkaline Aqueous Solution

20.02 g of the residual solution B and 10.04 g of a pH 12.8 sodium hydroxide solution were supplied into a 50 mL screw-capped bottle having a stirrer tip, stirred vigorously for 5 minutes and then allowed to stand for 2 minutes. As a result, two-layer separation of the mixed solution could be confirmed. The oil layer after separation was substantially free of aluminum and titanium.

### Comparative Example 6

The same experiment as conducted in Example 7 (3) was carried out except that 20.08 g of the residual solution B and 10.18 g of a pH 11.4 sodium hydroxide solution were used in place of 20.02 g of the residual solution B and 10.04 g of a pH 12.8 sodium hydroxide solution. As a result, a cloudy layer was observed present between the two layers. Since it was difficult to collect the oil layer, the metallic concentration was not able to be analyzed.

### Comparative Example 7

The same experiment as conducted in Example 7 (3) was carried out except that 20.06 g of the residual solution B and 10.04 g of a pH 13.1 sodium hydroxide solution were used in place of 20.02 g of the residual solution B and 10.04 g of a pH 12.8 sodium hydroxide solution. As a result, a cloudy layer was observed present between the two layers. Since it was difficult to collect the oil layer, the metallic concentration was not able to be analyzed.

### Example 8

In a well argon-substituted, stirrer-attached 200 mL autoclave, 5.5 mmol of methylaluminoxane (MMAO-3A made by TOSO Finechem, 1 M hexane solution) and 95 mL of toluene were mixed and then 3.2 MPa of ethylene was introduced. After further introducing 0.0005 mmol of a titanium complex (1.0 mM toluene solution) represented by the following formula (2), the mixture was stirred at 40° C. for 30 minutes. After the reaction, 1 mL of ethanol was introduced to terminate the reaction. After cooling to 0° C., ethylene was depressurized to obtain a reaction solution C. This reaction solution C contained 1.0 wt % of 1-hexene and 0.32 wt % of aluminum. Titanium was unmeasurable as it was below the detection limit.

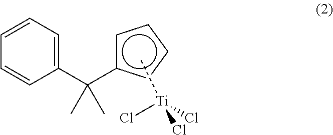

### Example 9

20.15 g of the reaction solution C and 10.01 g of a pH 12.7 sodium hydroxide solution were put into a 50 mL screw-capped bottle having a stirrer tip, stirred vigorously for 5 minutes and then allowed to stand for 2 minutes. As a result, it could be confirmed that the mixed solution separated into two layers. Substantially no aluminum and titanium were contained in the oil layer after separation.

The reaction solution C was distilled under normal pressure to separate 1-hexene. The solution which remained after separation of 1-hexene was called residual solution D. 0.1 wt % of 1-hexene was contained in the residual solution D. 20.29 g of the residual solution D and 10.01 g of a pH 12.4 sodium hydroxide solution were supplied into a 50 mL screw-capped bottle having a stirrer tip, stirred vigorously for 5 minutes and then allowed to stand for 2 minutes. As a result, it could be confirmed that the mixed solution separated into two layers. The oil layer after separation was substantially free of aluminum and titanium.

From the foregoing, it can be known that by contacting a solution containing metallic substances and 1-hexene or a solution containing metallic substances obtained by separating 1-hexene from the first-said solution and a solution containing a metal hydroxide and having a pH Of 12.0 or over, the oil-water separatability can be improved to facilitate removal of metallic substances.

1. A method of producing 1-hexene comprising,
step 1: a periodic table group IV transition metal compound, an organoaluminum compound and ethylene are brought into contact with each other in a solvent to obtain a solution containing 1-hexene; and
step 2: the solution obtained in step 1 is brought into contact with an aqueous solution containing a metal hydroxide and having a pH of 12.0 or higher.

2. The method of producing 1-hexene according to claim 1, wherein the step 2 comprises step 2′ of bringing the solution obtained in step 1 into contact with an aqueous solution containing a metal hydroxide and having a pH of 12,0 or higher to, cause a separation into a solution containing 1-hexene and a water layer, and
the method further comprises step 3 of separating 1-hexene from the solution containing 1-hexene obtained in step 2′.

3. The method of producing 1-hexene according to claim 1, wherein the step 2 comprises:
step 4 of separating at least part of 1-hexene from the solution obtained in step 1 to obtain 1-hexene or a solution containing 1-hexene and a residual solution from which at least part of 1-hexene has been separated; and
step 2″ of bringing the residual solution obtained in step 4 into contact with an aqueous solution containing a metal hydroxide and having a pH of 12.0 or higher to cause an oil-water separation.

4. The method of producing 1-hexene according to claim 1, wherein the periodic table group IV transition metal compound is titanium compound, zirconium compound or hafnium compound.

5. The method of producing 1-hexene according to claim 1, wherein the organoaluminum compound is a trialkylaluminum compound, an alkylaluminum halide compound, an alkylaluminum hydride compound, or an aluminoxane compound.

6. The method of producing 1-hexene according to claim 1, wherein the metal hydroxide is lithium hydroxide, sodium hydroxide, potassium hydroxide, rubidium hydroxide, cesium hydroxide, calcium hydroxide.
